(19) United States
(12) Patent Application Publication
(10) Pub. No.: US 2013/0225639 A1
(43) Pub. Date: Aug. 29, 2013

(54) ALTERING PHARMACOKINETICS OF PIRFENIDONE THERAPY

(71) Applicant: INTERMUNE, INC., (US)

(72) Inventors: Cynthia Y. Robinson, Burlingame, CA (US); Jeffery S. Loutit, Los Altos, CA (US); Michelle M. Freemer, Mill Valley, CA (US)

(73) Assignee: INTERMUNE, INC., Brisbane, CA (US)

(21) Appl. No.: 13/857,465

(22) Filed: Apr. 5, 2013

Related U.S. Application Data
(63) Continuation of application No. 13/028,827, filed on Feb. 16, 2011, now abandoned.
Application No. 13/028,827 is a continuation of application No. 12/020,068, filed on Jan. 25, 2008, now abandoned.
(60) Provisional application No. 60/938,850, filed on May 18, 2007.

(51) Int. Cl. A61K 31/4418

(52) U.S. Cl. CPC A61K 31/4418
USPC 514/345

(57) ABSTRACT

The invention relates to methods for reducing adverse events in patients receiving pirfenidone (5-methyl-1-phenyl-2-(1H)-pyridone) therapy.

### CROSS-REFERENCE TO RELATED APPLICATIONS

This application is a continuation of U.S. application Ser. No. 12/020,068, filed Jan. 25, 2008, which in turn claims the benefit of U.S. Provisional Application Ser. No. 60/938,850, filed May 19, 2007, each disclosure is incorporated by reference in its entirety.

### BACKGROUND

1. Technical Field

The invention relates to methods for decreasing adverse events associated with pirfenidone (5-methyl-1-phenyl-2-(1H)-pyridone) therapy.

2. Description of the Related Art

Pirfenidone is small drug molecule whose chemical name is 5-methyl-1-phenyl-2-(1H)-pyridone. It is a non-peptide synthetic molecule with a molecular weight of 185.23 daltons. Its chemical elements are expressed as C₁₂H₁₁NO, and its structure and synthesis are known. Pirfenidone is manufactured commercially and being evaluated clinically as a broad-spectrum anti-fibrotic drug. Pirfenidone has anti-fibrotic properties via: decreased TGF-β expression, decreased TNF-α expression, decreased PDGF expression, and decreased collagen expression. Several pirfenidone Investigational New Drug Applications (INDs) are currently on file with the U.S. Food and Drug Administration. Phase II human investigations are ongoing or have recently been completed for pulmonary fibrosis, renal glomerulosclerosis, and liver cirrhosis. There have been other Phase II studies that used pirfenidone to treat benign prostate hypertrophy, hypertrophic scarring (keloids), and rheumatoid arthritis.

Pirfenidone is being investigated for therapeutic benefits to patients suffering from fibrosis conditions such as Hermansky-Pudlak Syndrome (HPS) associated pulmonary fibrosis and idiopathic pulmonary fibrosis (IPF). Pirfenidone is also being investigated for a pharmacologic ability to prevent or remove excessive scar tissue found in fibrosis associated with injured tissues including that of lungs, skin, joints, kidneys, prostate glands, and livers. Published and unpublished basic and clinical research suggests that pirfenidone may safely slow or inhibit the progressive enlargement of fibrotic lesions, and prevent formation of new fibrotic lesions following tissue injuries.

It is understood that one mechanism by which pirfenidone exerts its therapeutic effects is modulating cytokine actions. Pirfenidone is a potent inhibitor of fibrogenic cytokines and TNF-α. It is well documented that pirfenidone inhibits excessive biosynthesis or release of various fibrogenic cytokines such as TGF-β1, bFGF, PDGF, and EGF. Zhang S et al., Australian and New England J Ophthalmology 26:S74-S76 (1998). Experimental reports also show that pirfenidone blocks the synthesis and release of excessive amounts of TNF-α from macrophages and other cells. Cain et al., Int'l J Immunopharmacology 20:685-695 (1998).

As an investigational drug, pirfenidone is provided in tablet and capsule forms principally for oral administration. Various formulations have been tested and adopted in clinical trials and other research and experiments. The most common adverse reactions or events associated with pirfenidone therapy include gastrointestinal upset, nausea, fatigue, somnolence, dizziness, headache, and photosensitivity rash. Many of these effects can interfere with everyday activities and quality of life. These effects appear to be dose related. The adverse reactions associated with pirfenidone therapy are exacerbated when pirfenidone is administered at these higher doses.

Currently, adverse events following administration of pirfenidone are alleviated by dose reduction or discontinuation of pirfenidone. In a recent study, for adverse events rated Grade 2 or worse, the dosage was reduced in a stepwise manner: from 9 tablets per day to 6 tablets per day and 6 tablets per day to 3 tablets per day. Azuma, A. et al., Am J Respir Crit Care Med 171:1040-47 (2005). If, after a period of 14 days of observation with reduced dosage, the adverse event persisted or increased, the dosage was further reduced by one more step—from 6 tablets per day to 3 tablets per day. If the adverse event persisted or increased despite reducing the dosage to 3 tablets per day, the study medication was discontinued.

There remains an unmet clinical need for a method of administering higher doses of pirfenidone to a patient in a manner that eliminates or minimizes adverse events, such as nausea, vomiting, gastrointestinal upset, drowsiness, dizziness, headache, somnolence, and other potentially dangerous side effects that can occur with pirfenidone therapy.

### SUMMARY

The invention disclosed herein is based on the unexpected finding that the administration of pirfenidone at or around the time food is consumed decreases the adverse events associated with the oral dosage form in humans. It is also unexpected that administration with food reduces the mean maximum plasma concentration of pirfenidone, thus reducing toxicity associated with a spike in drug concentration, without significantly affecting the total amount of drug absorbed.

In an embodiment, a method of reducing the likelihood of adverse events in a patient receiving pirfenidone therapy wherein the pirfenidone is in the form of a pharmaceutical composition is disclosed. The method comprises, for example, administering a therapeutically effective amount of pirfenidone to a patient with food.

In an embodiment, a method of reducing the likelihood of somnolence in a patient receiving pirfenidone therapy wherein the pirfenidone is in the form of a pharmaceutical composition is disclosed. The method comprises, for example, administering a therapeutically effective amount of pirfenidone to the patient with food.

In an embodiment, a method of reducing the likelihood of nausea in a patient receiving pirfenidone therapy wherein the pirfenidone is in the form of a pharmaceutical composition is disclosed. The method comprises, for example, administering a therapeutically effective amount of pirfenidone to the patient with food.

In an embodiment, a method of reducing the likelihood of headaches in a patient receiving pirfenidone therapy wherein the pirfenidone is in the form of a pharmaceutical composition is disclosed. The method comprises, for example, administering a therapeutically effective amount of pirfenidone to the patient with food.

In some embodiments, the likelihood of one or more adverse effects is reduced. For example, in some embodiments, the likelihood of nausea and somnolence is reduced. In other embodiments, the likelihood of nausea and headaches is reduced. In still other embodiments, the likelihood of somnolence and headaches is reduced. In some embodiments, the likelihood of nausea, somnolence and headaches is reduced.

In some embodiments, the methods comprise administering pirfenidone to a patient, wherein the administering comprises providing pirfenidone in about 100 milligrams to about 400 milligrams per unit dosage form. In some embodiments, the administering comprises providing one or more unit dosage forms one or more times per day to the patient. In an embodiment, the administering comprises providing one or more capsules comprising pirfenidone one or more times per day to the patient. In an embodiment, the administering comprises providing one or more capsules comprising about 267 mg of pirfenidone one or more times per day to the patient.

In some embodiments, the administering comprises providing greater than 1800 mg/day of pirfenidone to the patient. In some embodiments, the administering comprises providing from about 2000 mg/day to about 4000 mg/day of pirfenidone to the patient. In some embodiments, the administering comprises providing from about 2200 mg/day to about 4000 mg/day of pirfenidone to the patient. In some embodiments, the administering comprises providing from about 2400 mg/day to about 4000 mg/day of pirfenidone to the patient. In an embodiment, the administering comprises providing about 2403 mg/day of pirfenidone to the patient.

In some embodiments, the food is a solid food with sufficient caloric and fat content that it is not rapidly dissolved and absorbed in the stomach. Thus, in some embodiments, the food is a meal, for example, breakfast, lunch or dinner.

In some embodiments, the therapeutically effective amount of pirfenidone is administered to the patient between about 1 hour prior to about 2 hours after eating a meal. In some embodiments, the pirfenidone is administered to the patient within about 30 minutes, about 15 minutes of consuming food.

In some embodiments, the methods disclosed herein further comprise providing information to prescribing physicians and patients receiving pirfenidone therapy useful for decreasing adverse events when taking pirfenidone. In preferred embodiments, the methods further comprise advising a patient to take pirfenidone with food. In some embodiments, the methods further comprise advising a patient to take pirfenidone with food to avoid and/or minimize adverse events associated with pirfenidone therapy.

In some embodiments, the methods include providing the composition to the patient in a container associated with printed labeling advising that the administration with food results in a reduction in the likelihood of adverse events. In some embodiments, the methods include providing the pharmaceutical composition to the patient in a container associated with printed labeling advising the patient that the pharmaceutical composition is to be administered between about 1 hour prior to consuming food to about 2 hours after consuming food. In some embodiments, the methods include providing the pharmaceutical composition to the patient in a container associated with printed labeling advising the patient that the pharmaceutical composition is to be administered at substantially the same time as consuming food.

Another embodiment provides an article of manufacture or a kit comprising a container, wherein the container holds a pharmaceutical composition comprising pirfenidone in unit dosage form, and printed labeling instructions advising of the varying side effects when the composition is taken with and without food. In some embodiments, the printed instructions advise the patient to take the composition with food if stomach upset or somnolence occurs.

In some embodiments, the printed instructions further advise the patient that the administration of the composition with food results in a reduction in the likelihood of adverse events. In some embodiments, the printed instructions advise the patient to take the composition between about 1 hour prior to consuming food to about 2 hours after consuming food. In some embodiments, the printed instructions advise the patient to take the composition at substantially the same time as consuming food. In some embodiments, the printed instructions advise the patent to take the composition between about 30 minutes prior to about 2 hours after consuming food. In some embodiments, the printed instructions advise the patient to take the composition immediately after the consumption of food up to 1 hour after said consumption. In some embodiments, the printed instructions advise the patient to take the composition with a meal.

In some embodiments, the printed instructions advise the patient to take one or more of the capsules twice per day. In some embodiments, the printed instructions advise the patient to take one or more capsules three times per day.

In another embodiment, a method for providing pirfenidone therapy to a patient is disclosed, comprising providing a therapeutic dose of pirfenidone to the patient, and advising the patient to take the pirfenidone with food.

Another disclosed embodiment is a method for providing pirfenidone therapy to a patient, comprising providing a therapeutic dose of pirfenidone to the patient, and advising the patient that consuming the pirfenidone with food may reduce the incidence of adverse events resulting from pirfenidone therapy.

Also disclosed is a method for providing pirfenidone therapy to a patient, comprising providing a therapeutic dose of pirfenidone (usually contained within a pharmaceutical composition) to the patient; and advising the patient that consuming the pirfenidone with food significantly reduces mean maximum plasma concentration (Cₘₐₓ)of pirfenidone and/or significantly increases (makes longer) the mean absorption half life (t_{1/2, abs}) of pirfenidone in comparison to consuming the pirfenidone without food. In certain embodiments, the pirfenidone is consumed within one hour or 30 minutes of the food consumption. In some embodiments, the pirfenidone is consumed at the same time as the food consumption. In other embodiments, the pirfenidone is consumed during the time period from one hour prior to food consumption to two hours after food consumption.

In some embodiments, the patient may be advised that consuming pirfenidone with food (e.g., about 801 mg, 1602 mg, 2403 mg or more) significantly reduces mean maximum plasma concentration of pirfenidone such that the ratio of the average Cₘₐₓ for the fed patient to the average Cₘₐₓ of the fasted patient (C_{max(fed)}:C_{max(fasted)}) ranges from about 0.3 to about 0.8, about 0.35 to about 0.75, about 0.4 to about 0.7, about 0.4 to about 0.6, about 0.4 to about 0.5, or about 0.45 to about 0.55. In a specific embodiment, the average Cₘₐₓ of pirfenidone is reduced from 15724 ng/mL without food to 7874 ng/mL with food.

Additionally or alternatively, in some embodiments, the patient may be advised that consuming pirfenidone with food (e.g., about 801 mg, 1602 mg, 2403 mg or more) significantly increases mean absorption half life of the pirfenidone such that the ratio of mean t_{1/2, abs} of the fed patient to mean t_{1/2, abs} of the fasted patient (t_{1/2, abs(fed)}:t_{1/2, abs(fasted)}) ranges from about 1.5 to about 5, about 1.75 to about 4.5, about 2 to about 4, about 2.5 to about3.5, about 2.75 to about 3.5, or about 2.75 to 3.25. In a specific embodiment, the mean t_{1/2, abs} increases from 0.572 hours without food to 1.78 hours with food.

In some embodiments, the patient may be advised that consuming pirfenidone with food maintains at least 80% of the overall mean absorption of pirfenidone in comparison to consuming pirfenidone without food, as measured by the Area Under the Curve (AUC) of an absorption profile. In specific embodiments, the total absorption of pirfenidone consumed with food is at least 85% or at least 88% that of pirfenidone consumed without food, as measured by AUC.

In all the embodiments, it is contemplated that the patient may be advised in writing or orally, and that the written information may be contained (for example) in a label, a sticker, a product insert, product information, or prescribing information.

In related embodiments, the invention provides a method for administering pirfenidone to a human patient in need thereof, e.g. a patient suffering from pulmonary fibrosis, comprising administering a pharmaceutical composition comprising a therapeutic dose of pirfenidone with food to the patient, wherein the mean maximum plasma concentration (Cₘₐₓ) of pirfenidone is significantly reduced and/or the mean absorption half life (t_{1/2, abs}) of pirfenidone is significantly longer. In some embodiments, the ratio of the average Cₘₐₓ for the fed patient to the average Cₘₐₓ of the fasted patient (C_{max(fed)}:C_{max(fasted)}) ranges from about 0.3 to about 0.8, about 0.35 to about 0.75, about 0.4 to about 0.7, about 0.4 to about 0.6, about 0.4 to about 0.5, or about 0.45 to about 0.55, or about 0.5, and/or wherein the ratio of mean t_{1/2, abs} of the fed patient to mean t_{1,2, abs} of the fasted patient (t_{1/2, abs(fed)}:t_{1/2, abs (fasted)}) ranges from about 1.5 to about 5, about 1.75 to about 4.5, about 2 to about 4, about 2.5 to about 3.5, about 2.75 to about 3.5, or about 2.75 to 3.25, or about 3.

### BRIEF DESCRIPTION OF THE DRAWING

FIGS. 1A and 1B are graphs summarizing pharmacokinetic data for fasted and fed patients, where FIG. 1A shows the mean Cₘₐₓ of fasted patients is 15724 ng/mL and the Cₘₐₓ of fed patients is 787 ng/mL and FIG. 1B shows the mean AUC of fasted patients is 72.7 mg h/L (standard deviation of 19.9) and the mean AUC of fed patients is 64.6 mg h/L (standard deviation of 21.3).

### DETAILED DESCRIPTION

The most common adverse reactions or events reported during pirfenidone therapy include gastrointestinal upset, nausea, fatigue, somnolence, dizziness, headache, and photosensitivity rash. Many of these effects can interfere with everyday activities and quality of life. These effects appear to be dose related, and are typically alleviated following dose-reduction or discontinuation of pirfenidone therapy. It is a novel discovery that administration of pirfenidone at or around the time food is consumed alleviates adverse events associated with the oral dosage form of pirfenidone in humans. In some embodiments, dosage may also be reduced.

As used herein, the terms “adverse event” and “adverse reactions” refer to any unfavorable, harmful, or pathologic change in a patient receiving pirfenidone therapy as indicated by physical signs, symptoms, and/or clinically significant laboratory abnormalities that occur in a patient during the treatment and post-treatment period, regardless of suspected cause. This definition includes the following: intercurrent illness; injuries; exacerbation of pre-existing conditions; adverse events occurring as a result of product withdrawal, abuse, or overdose; and a change in a laboratory variable if considered by the attending physician to be clinically significant or if it caused (or should have caused) the clinician to reduce or discontinue the use of the product or initiate a non-protocol therapy or procedure.

The term “pirfenidone” includes salts thereof.

As used herein, the terms “with food” or “fed conditions” are defined to mean, in general, the condition of having consumed food during the period between from about 1 hour prior to the administration of pirfenidone to about 2 hours after the administration of pirfenidone. In some cases, the pirfenidone is administered within about 30 minutes or 1 hour of the food, and in other cases, the pirfenidone is administered at the same time as the food, e.g., with a meal. In some embodiments, the food is a solid food with sufficient bulk and fat content that it is not rapidly dissolved and absorbed in the stomach. Preferably, the food is a meal, such as breakfast, lunch, or dinner. In some embodiments, the food is at least about 100 calories, about 200 calories, about 250 calories, about 300 calories, about 400 calories, about 500 calories, about 600 calories, about 700 calories, about 800 calories, about 900 calories, about 1000 calories, about 1250 calories, about 1500 calories.

The terms “without food,” “fasted,” “fasted conditions,” or “on an empty stomach” are defined to mean the condition of not having consumed food within the time period of about 1 hour prior to the administration of pirfenidone to about 2 hours after the administration of pirfenidone. In some embodiments, food has not been consumed for about 10 hours, about 8 hours, about 6 hours, about 4 hours, about 2 hours prior to administration of pirfenidone.

The terms “patient” or “subject” refers to a human patient.

The methods disclosed herein include administering pirfenidone to a patient with food. The pirfenidone can be administered any time of day with food. For example, in some embodiments, the food can be consumed at any time during the period between from about 2 hours prior to the administration of pirfenidone to about 2 hours after the administration of pirfenidone. In some embodiments, the food can be consumed within the time period of about 2 hours, about 1.5 hours, about 1 hour, about 45 minutes, about 30 minutes, about 15 minutes, about 10 minutes, or about 5 minutes prior to the administration of pirfenidone. In some embodiments, the food can be consumed within the time period of about 5 minutes, about 10 minutes, about 15 minutes, about 30 minutes, about 45 minutes, about 1 hour, about 1.5 hours, or about 2 hours after the administration of pirfenidone. In some embodiments, the administration of pirfenidone to the patient is immediately after the consumption of food (e.g., within about 1 minute after food consumption) up to about 1 hour after food consumption. In some embodiments, pirfenidone is administered at substantially the same time as the consumption of the food.

In some embodiments, an effective daily intake of pirfenidone is greater than 1800 mg/day. In some embodiments, an effective daily intake of pirfenidone is about 2000 mg to about 4005 mg per day. In some embodiments, an effective daily intake of pirfenidone is about 2200 mg to about 4000 mg per day. In some embodiments, an effective daily intake of pirfenidone is about 2400 to about 3600 mg per day. In some embodiments, an effective daily intake of pirfenidone is about 2403 mg/day.

In an embodiment, pirfenidone is administered to the subject in a unit dosage form comprising about 100 to about 400 mg of pirfenidone per unit. In an embodiment, pirfenidone is administered to the subject in a unit dosage form comprises about 267 mg of pirfenidone per capsule. In preferred embodiments, the unit dosage form is a capsule.

The dosing may be once or twice or three times daily, with one or more units per dose. In some embodiments, the effective daily intake of pirfenidone is administered as one, two, three, four, five, six, or more doses administered separately at appropriate intervals throughout the day. In some embodiments, each dose comprises one, two, three or more unit dosage forms. For example, in some embodiments, one or more units are administered to the subject one or more times per day. In some embodiments, one or more units are administered to the subject twice per day. In some embodiments, one or more units are administered to the subject three times per day. In some embodiments, 3 units are administered three times per day. In some embodiments, pirfenidone is administered as multiple doses spaced throughout the day and each dose comprises a therapeutically effective amount of pirfenidone. In some embodiments, pirfenidone is administered with food once per day.

As used herein, the term “unit dosage form,” refers to physically discrete units suitable as unitary dosages for human and animal subjects, each unit containing a predetermined quantity of pirfenidone calculated in an amount sufficient to produce the desired effect in association with a pharmaceutically acceptable diluent, carrier or vehicle. In some embodiments, the unit dosage form is, for example, a pill, capsule, or tablet. In some embodiments, the unit dosage form is a capsule. In some embodiments, the amount of pirfenidone in a unit dosage form is about 100 mg to about 1800 mg, or about 200 mg to about 900 mg, or about 100 mg to about 400 mg. In an embodiment, the unit dosage form comprises about 267 mg of pirfenidone and is in the form of a capsule. In some embodiments, two or three capsules, each of which comprises about 267 mg of pirfenidone, are administered to the patient once, twice or three times per day (e.g., a total daily intake of about 534 mg/day to about 2403 mg/day).

In some embodiments, the methods include administering a therapeutically acceptable amount of pirfenidone. The terms “therapeutically effective amount” and “prophylactically effective amount,” as used herein, refer to an amount of pirfenidone sufficient to treat, ameliorate, or prevent the identified disease or condition, or to exhibit a detectable therapeutic, prophylactic, or inhibitory effect. The effect may be detected by any means known in the art. In some embodiments, the precise effective amount for a subject can depend upon the subject's body weight, size, and health; the nature and extent of the condition; and the therapeutic or combination of therapeutics selected for administration. Therapeutically and prophylactically effective amounts for a given situation may be determined by routine experimentation that is within the skill and judgment of the clinician.

The therapeutically or prophylactically effective amount of pirfenidone may be estimated initially either in cell culture assays or in animal models, usually rats, mice, rabbits, dogs, or pigs. The animal model may also be used to determine the appropriate concentration range and route of administration. Such information may then be used to determine useful doses and routes for administration in humans.

Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it may be expressed as the ratio, ED₅₀/LD₅₀. Pharmaceutical compositions that exhibit large therapeutic indices are preferred. However, the pharmaceutical compositions that exhibit narrow therapeutic indices are also within the scope of the embodiments. The data obtained from cell culture assays and animal studies may be used in formulating a range of dosage for human use. The dosage contained in such compositions is preferably within a range of circulating concentrations that include an ED₅₀ with little or no toxicity. The dosage may vary within this range depending upon the dosage form employed, sensitivity of the patient, and the route of administration.

More specifically, the maximum plasma concentrations (Cₘₐₓ) of pirfenidone may range from about 65 μM to about 115 μM, or about 75 μM to about 105 μM, or about 85 μM to about 95 μM, or about 85 μM to about 90 μM depending upon the route of administration. For fed conditions, the Cₘₐₓ is typically lower that the Cₘₐₓ of pirfenidone under fasted conditions. In some embodiments, the ratio of C_{max(fed)} to C_{max(fasted)} is about 0.3 to about 0.8, about 0.35 to about 0.7, about 0.4 to about 0.65. about 0.4 to about 0.6, about 0.45 to about 0.65, or about 0.45 to about 0.55. In specific embodiments, the ratio of C_{max(fed)} to C_{max(fasted)} is about 0.5.

The absorption half life of pirfenidone when administered under fed conditions (t_{1/2, abs (fed)}) is typically longer than the absorption half life of pirfenidone when administered under fasted conditions (t_{1/2, abs (fasted)}). In some embodiments, the ratio of t_{1/2, abs (fed)} to t_{1/2, abs (fasted)} (t_{1/2, abs (fed)}:t_{1/2, abs (fasted)}) is about 1.5 to about 5, about 2 to about 4, or about 2.5 to about 3.5, about 2.75 to about 3.5, or about 2.75 to 3.25. In specific embodiments, t_{1/2, abs (fed)}:t_{1/2, abs (fasted)} is about 2, about 2.5, about 3, about 3.5, about 4, about 4.5, or about 5. In a specific embodiment, the t_{1/2 abs (fed)} is about 1.78 hours and the t_{1/2, abs (fasted)} is about 0.572 hours.

The total absorption of pirfenidone under fed or fasted conditions can also be determined by comparing the area under the curve (AUC) of the absorption curves. In some embodiments, the AUC_{fed} is at least about 80%, 82%, 84%, 85%, 86%, 87%, 88%, 89%, 90%, 91%, 92%, 93% or at least about 94% that of AUC_{fasted}.

In general the daily intake will be in the range of about 100 mg/day to about 10 g/day, or about 200 mg to about 5 g/day, or about 400 mg to about 3 g/day, or about 500 mg to about 2 g/day, in single, divided, or continuous doses for a patient weighing between about 40 to about 100 kg (which doses may be adjusted for patients above or below this weight range, particularly children under 40 kg). Generally the daily intake will be in the range of about 25 mg/kg to about 200 mg/kg of body weight per day. In some embodiments, the maximum daily intake of pirfenidone is 4 g/day.

The exact dosage will typically be determined by the practitioner, in light of factors related to the subject that requires treatment. Dosage and administration are generally adjusted to provide sufficient levels of pirfenidone or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The specifications for the unit dosage forms described herein depend on the particular dose employed and the effect to be achieved, and the pharmacodynamics associated with pirfenidone in the host.

The decrease in duration or number of adverse events in a patient receiving pirfenidone therapy can be evidenced in any suitable manner. Desirably, the oral administration of pirfenidone with food results in a reduction in the frequency and/or severity of adverse events as evidenced by a review of adverse events following administration of pirfenidone as compared to the administration of pirfenidone without food.

In some embodiments, pirfenidone is provided to a patient in a container associated with prescribing information that advises the patient to take the pharmaceutical composition with food, and in some embodiments further advises the patient that taking the composition with food results in a reduction in the duration, likelihood, and/or severity of adverse events associated with pirfenidone therapy. In some embodiments, the prescribing information advises the patient to take the composition with food if stomach upset and/or somnolence occurs.

In some embodiments, the methods can include identifying a subject at risk for or suffering from an adverse event associated with pirfenidone therapy and administering a therapeutically effective amount of pirfenidone with food. The term “at risk for or suffering from” as used herein, refers to subjects having previously experienced, or currently experiencing, or having a high probability of experiencing an adverse event associated with pirfenidone therapy. Methods for identifying a subject at risk for or suffering from such adverse events are known in the art.

In an embodiment, the methods include identifying a patient who could benefit from the methods disclosed herein. In some embodiments, the methods described herein include identifying a subject who has experienced or is experiencing an adverse event, such as gastrointestinal symptoms, somnolence, and/or headache, following administration of pirfenidone. Identifying such subjects may be accomplished by any means that indicates a subject who may benefit from the methods disclosed herein, for example, by clinical diagnosis, laboratory testing, or any other means known to one of skill in the art, including any combination of means for identification.

It will be appreciated that the methods described herein include preventing, alleviating, and/or minimizing the duration and/or severity of adverse events associated with pirfenidone therapy.

In an embodiment, the methods disclosed herein result in a reduction in the likelihood of nausea in patients receiving pirfenidone therapy with food (fed) as compared to patients receiving pirfenidone therapy without food (fasted). Preferably, the likelihood of nausea of a fed population is reduced by at least about 25% relative to the likelihood of nausea of a fasted population; more preferably, the likelihood of nausea is reduced by at least about 30%; more preferably, reduced by at least about 33%; more preferably, reduced by at least about 40%; more preferably, reduced by at least about 50%; more preferably, reduced by at least about 60%; even more preferably, reduced by at least 70%; and most preferably, reduced by at least about 75%. Likelihood of nausea may be measured by any reproducible means of measurement.

In an embodiment, the methods disclosed herein result in a reduction in the likelihood of somnolence in patients receiving pirfenidone therapy with food (fed) as compared to patients receiving pirfenidone therapy without food (fasted). Preferably, the likelihood of somnolence of a fed population is reduced by at least about 25% relative to the likelihood of somnolence of a fasted population; more preferably, the likelihood of somnolence is reduced by at least about 30%; more preferably, reduced by at least about 33%; more preferably, reduced by at least about 40%; more preferably, reduced by at least about 50%; more preferably, reduced by at least about 60%; even more preferably, reduced by at least 70%; and most preferably, reduced by at least about 75%. Likelihood of somnolence may be measured by any reproducible means of measurement.

In an embodiment, the methods disclosed herein result in a reduction in the likelihood of headache in patients receiving pirfenidone therapy with food (fed) as compared to patients receiving pirfenidone therapy without food (fasted). Preferably, the likelihood of headache of a fed population is reduced by at least about 25% relative to the likelihood of headache of a fasted population; more preferably, the likelihood of headache is reduced by at least about 30%; more preferably, reduced by at least about 33%; more preferably, reduced by at least about 40%; more preferably, reduced by at least about 50%; more preferably, reduced by at least about 60%; even more preferably, reduced by at least 70%; and most preferably, reduced by at least about 75%. Likelihood of headache may be measured by any reproducible means of measurement.

In an embodiment, the methods disclosed herein result in a reduction in the likelihood of dizziness in patients receiving pirfenidone therapy with food (fed) as compared to patients receiving pirfenidone therapy without food (fasted). Preferably, the likelihood of dizziness of a fed population is reduced by at least about 25% relative to the likelihood of dizziness of a fasted population; more preferably, the likelihood of dizziness is reduced by at least about 30%; more preferably, reduced by at least about 33%; more preferably, reduced by at least about 40%; more preferably, reduced by at least about 50%; more preferably, reduced by at least about 60%; even more preferably, reduced by at least 70%; and most preferably, reduced by at least about 75%. Likelihood of dizziness may be measured by any reproducible means of measurement.

As described elsewhere herein, pirfenidone may be formulated in pharmaceutical compositions, if desired, and may be administered by any route that permits treatment of the disease or condition. A preferred route of administration is oral administration. Administration may take the form of single dose administration, or pirfenidone may be administered over a period of time, either in divided doses or in a continuous-release formulation or administration method (e.g., a pump). However pirfenidone is administered to the subject, the amount administered and the route of administration chosen should be selected to permit efficacious treatment of the disease condition.

### Pharmaceutical Compositions

While it is possible for pirfenidone to be administered alone, it may be preferable to formulate pirfenidone as pharmaceutical compositions. As such, in yet another aspect, pharmaceutical compositions useful in the methods of the invention are provided. More particularly, the pharmaceutical compositions described herein may be useful, inter alia, for treating or preventing neutropenia. A pharmaceutical composition is any composition that may be administered in vitro or in vivo or both to a subject in order to treat or ameliorate a condition. In a preferred embodiment, a pharmaceutical composition may be administered in vivo. A mammal includes any mammal, such as by way of non-limiting example, cattle, pigs, sheep, goats, horses, camels, buffalo, cats, dogs, rats, mice, and humans. A highly preferred subject mammal is a human.

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8. More particularly, the pharmaceutical compositions may comprise a therapeutically or prophylactically effective amount of pirfenidone, together with one or more pharmaceutically acceptable excipients. Optionally, the pharmaceutical compositions may comprise a combination of pirfenidone and a second active ingredient useful in the treatment or prevention of the disease or condition being treated.

Formulations, e.g., for parenteral or oral administration, are most typically solids, liquid solutions, emulsions or suspensions, while inhalable formulations for pulmonary administration are generally liquids or powders, with powder formulations being generally preferred. A preferred pharmaceutical composition may also be formulated as a lyophilized solid that is reconstituted with a physiologically compatible solvent prior to administration. Alternative pharmaceutical compositions may be formulated as syrups, creams, ointments, tablets, capsules and the like.

The term “pharmaceutically acceptable excipient” refers to an excipient for administration of a pharmaceutical agent, such as the compounds described herein. The term refers to any pharmaceutical excipient that may be administered without undue toxicity. Pharmaceutically acceptable excipients may include, for example, inactive ingredients such as disintegrators, binders, fillers, and lubricants used in formulating pharmaceutical products.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

Disintegrators include, for example, agar-agar, algins, calcium carbonate, carboxmethylcellulose, cellulose, clays, colloid silicon dioxide, croscarmellose sodium, crospovidone, gums, magnesium aluminium silicate, methylcellulose, polacrilin potassium, sodium alginate, low substituted hydroxypropylcellulose, and cross-linked polyvinylpyrrolidone hydroxypropylcellulose, sodium starch glycolate, and starch.

Binders include, for example, microcrystalline cellulose, hydroxymethyl cellulose, hydroxypropylcellulose, and polyvinylpyrrolidone.

Fillers include, for example, calcium carbonate, calcium phosphate, dibasic calcium phosphate, tribasic calcium sulfate, calcium carboxymethylcellulose, cellulose, dextrin derivatives, dextrin, dextrose, fructose, lactitol, lactose, magnesium carbonate, magnesium oxide, maltitol, maltodextrins, maltose, sorbitol, starch, sucrose, sugar, and xylitol.

Lubricants include, for example, agar, calcium stearate, ethyl oleate, ethyl laureate, glycerin, glyceryl palmitostearate, hydrogenated vegetable oil, magnesium oxide, magnesium stearate, mannitol, poloxamer, glycols, sodium benzoate, sodium lauryl sulfate, sodium stearyl, sorbitol, stearic acid, talc, and zinc stearate.

The pharmaceutical compositions described herein may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed. To those skilled in the pharmaceutical research and manufacturing, it is generally known that tablet formulations permit generous additions of inactive ingredients including excipients and coating substances, and a high percentage of fillers. However, the addition of inactive ingredients may limit the amount of active ingredients carried in each tablet.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil. Capsules may allow for inclusion of a larger amount of binders, instead of fillers as used more in tablets. The capsule shell may be made of hard gelatin in an embodiment. The shell may be clear or opaque, white or with color in various embodiments. In an embodiment, the capsule is size 1. Other sizes may be adopted in alternative embodiments. The benefits of using capsules include their slender shape, which make them easy to swallow and their ability to effectively mask unpleasant taste and/or odor associated with pirfenidone, resulting in higher patient satisfaction and greater patient compliance with pirfenidone therapy dosing regimens.

In another embodiment, pharmaceutical compositions may be formulated as suspensions comprising pirfenidone in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions may be in the form of a sterile injectable preparation, such as a sterile injectable aqueous emulsion or oleaginous suspension. This emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids such as oleic acid may likewise be used in the preparation of injectables.

To obtain a stable water-soluble dose form of a pharmaceutical composition, pirfenidone may be dissolved in an aqueous solution of an organic or inorganic acid, such as 0.3 M solution of succinic acid, or more preferably, citric acid. Pirfenidone may be dissolved in a suitable co-solvent or combination of co-solvents. Examples of suitable co-solvents include alcohol, propylene glycol, polyethylene glycol 300, polysorbate 80, glycerin and the like in concentrations ranging from about 0 to about 60% of the total volume. In an embodiment, pirfenidone is dissolved in DMSO and diluted with water.

The pharmaceutical composition may also be in the form of a solution of pirfenidone in an appropriate aqueous vehicle, such as water or isotonic saline or dextrose solution. Also included in the definition of pirfenidone are compounds which have been modified by substitutions or additions of chemical or biochemical moieties to pirfenidone which make them more suitable for delivery (e.g., increase solubility, bioactivity, palatability, decrease adverse reactions, etc.), for example by esterification, glycosylation, PEGylation, etc.

In a preferred embodiment, pirfenidone may be formulated for oral administration in a lipid-based formulation suitable for low solubility compounds. Lipid-based formulations may generally enhance the oral bioavailability of pirfenidone.

As such, a preferred pharmaceutical composition comprises a therapeutically or prophylactically effective amount of pirfenidone, together with at least one pharmaceutically acceptable excipient selected from the group consisting of—medium chain fatty acids or propylene glycol esters thereof (e.g., propylene glycol esters of edible fatty acids such as caprylic and capric fatty acids) and pharmaceutically acceptable surfactants such as polyoxyl 40 hydrogenated castor oil.

In an alternative preferred embodiment, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. A particularly preferred cyclodextrin solubility enhancer is hydroxypropyl-o-cyclodextrin (BPBC), which may be added to any of the above-described compositions to further improve the aqueous solubility characteristics of pirfenidone. In an embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of pirfenidone in the composition.

A pharmaceutical composition preferably contains a total amount of pirfenidone sufficient to achieve an intended therapeutic effect. The total amounts of pirfenidone that may be combined with the carrier materials to produce a unitary dosing form will vary depending upon the host treated and the particular mode of administration. Preferably, the compositions are formulated so that a daily intake of between 0.01 to 100 mg/kg body weight/day of pirfenidone is administered to a subject receiving the compositions.

In an embodiment, the composition is provided in the form of a capsule wherein by weight, 2-10% of the capsule is disintegrator, 2-30% is binder, 2-30% is filler, and 0.3-0.8% is lubricant. A multitude of substances may be suitably included as disintegrator, binder, filler, and lubricant. One example is to use magnesium stearate as lubricant, microcrystalline cellulose as binder, and croscarmellose as disintegrator. In an embodiment, the capsule formulation further includes povidone. By weight povidone may constitute 1-4% of the capsule. For example, in an embodiment of the invention, the composition is formulated as a capsule comprising 82.15% pirfenidone, 8.15% croscarmellose sodium, 7.38% microcrystalline cellulose, 1.85% povidine, USP, EP, and 0.46% magnesium stearate.

It is to be understood that the description, specific examples and data, while indicating exemplary embodiments, are given by way of illustration and are not intended to limit the various embodiments of the present disclosure. All references cited herein for any reason, are specifically and entirely incorporated by reference. Various changes and modifications within the present disclosure will become apparent to the skilled artisan from the description and data contained herein, and thus are considered part of the various embodiments of this disclosure. Individual embodiments may specifically include or exclude any such alternatives.

### EXAMPLES

### Example 1

### Single Dose Study

A study was designed to evaluate the effect of food, antacids, and food taken with antacids on adverse events associated with pirfenidone use. The trial was conducted as a randomized, open-label, four-treatment crossover, with a single dose for each treatment period and a 2-day washout period between study treatments. 16 healthy adults between the ages of 50 and 79 years having body mass indices between 18 and 30 (inclusive) were enrolled and completed all 4treatment arms. The treatment arms were as follows:

A) pirfenidone alone (Fasted);

B) pirfenidone within 1 minute following a dose of antacid (20 mL Mylanta® Maximum Strength Liquid) (Fasted+Antacid);

C) pirfenidone 5 minutes after completing a standard meal (Fed); and

D) pirfenidone 5 minutes after completing a standard meal, then within 1 minute, followed by a dose of antacid (Fed+Antacid).

All subjects were admitted to the clinic for clinical evaluation the day prior to receiving the first dose of the study medication and remained confined for 13 days. Following an overnight fast of at least 10 hours (h), subjects were randomized to one of the above-described 4 treatment sequences. Each dose of pirfenidone (3×267 mg capsules) was administered orally with 240 mL room temperature water on days 1, 4, 7, and 10 in the morning, following an overnight fast of at least 10 hours. The washout period between treatments was 2 days. A standard meal consisted of 2 fried eggs, 2 strips of bacon, 2 slices of toast, 2 pats of butter, hash brown potatoes (4 oz), and whole milk (8 oz).

On each study day, adverse events were reviewed with the patients as follows. Before administration of the first dose of the study drug, study site personnel noted the occurrence and nature of each subject's medical condition(s). During the trial, site personnel again noted any change in the condition(s) or occurrence and nature of any adverse events. The severity of adverse events were graded based on the Modified Common Toxicity Criteria and the relationship of the study drug to the adverse event determined.

Of subjects receiving pirfenidone therapy without food, 9 (or 56.3%) experienced an adverse event. Of subjects receiving pirfenidone therapy within 1 minute following a dose of antacid, 9 (56.3%) experienced an adverse event. In comparison, of subjects receiving pirfenidone therapy with food, 5 (31.3%) experienced an adverse event, and 4 (25%) subjects receiving pirfenidone therapy with food and antacid experienced an adverse event. In total, 12 subjects (75%) had at least 1 adverse event. In 8 of these 12 subjects (75%), the adverse events were mild. The most common adverse events were nausea (7 of 16 or 44%), dizziness (6 of 16 or 38%), and somnolence (4 of 16 or 25%).

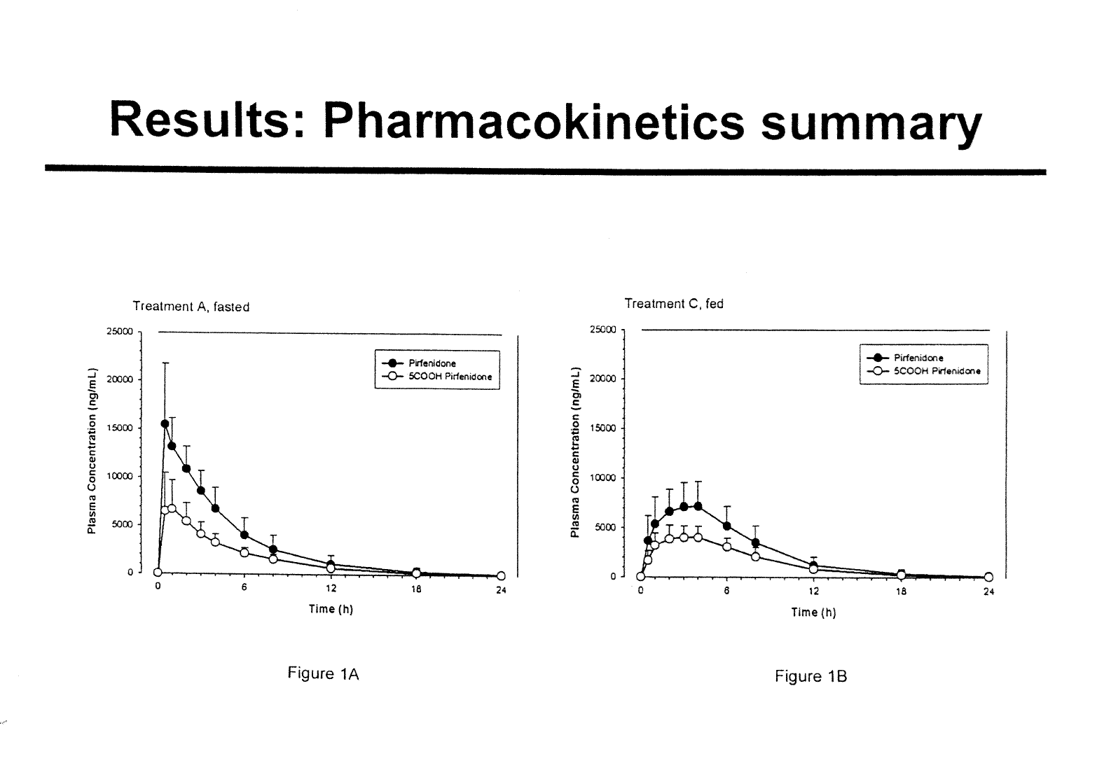

Of 7 subjects who reported nausea, 5 of 16 (31%) were fasted compared to only 2 of 16 (13%) fed subjects. Of 5 subjects reporting dizziness, 4 of 16 (25%) were fasted, while only 1 of 16 (6%) was fed. Of 3 subjects reporting somnolence, 2 of 16 (13%) were fasted and 1 of 16 (6%) were fed. Of 3 subjects reporting headaches, 2 of 16 (13%) were fasted and 1 of 16 (6%) were fed. Pharmacokinetic data for fed and fasted patients are summarized in FIGS. 1A and 1B.

### Example 2

### Multiple Dose Study

A second study was designed to examine incidences of adverse events on multiple ascending daily doses of pirfenidone. The trial was conducted as an open-label, escalating-dose study with no washout period between dose escalations. 25 healthy adults between the ages of 45 and 79 (inclusive) having body mass indices between 18 and 30 (inclusive) were enrolled. 22 adults completed the treatment. Each volunteer received from 801 mg/day to 4005 mg/day of pirfenidone divided into three equal doses as follows:

Days 1-2: 1 capsule three times a day (TID) (801 mg total daily dose (TDD))

Day 3: 1 capsule in the morning (˜0800) (267 mg TDD)

Days 4-5: 2 capsules TID (1602 mg TDD)

Day 6: 2 capsules in the morning (534 mg TDD)

Days 7-8: 3 capsules TID (2403 mg TDD)

Day 9: 3 capsules in the morning (801 mg TDD)

Days 10-11: 4 capsules TID (3204 mg TDD)

Day 12: 4 capsules in the morning (1068 mg TDD)

Days 13-14: 5 capsules TID (4005 mg TDD)

Day 15: 5 capsules in the morning (1335 mg TDD)

All subjects were admitted to the clinic for clinical evaluation the day before the first dose of study medication and remained confined for 17 days. Each subject received all dose levels of the study drug unless adverse events or the clinical judgment of the trial physician precluded the continuation of treatment. All doses were taken with 240 mL room temperature water and with food (excluding grapefruit juice). TID doses times were ˜0800, ˜1200, and ˜1800.

23 (92%) of all subjects had at least one adverse event. In 22 of these 23 subjects (96%), the adverse events were mild. The most common adverse events were nausea (9 of 25 or 36%), headache (7 of 25 or 28%), and somnolence (8 of 25 or 32%).

What is claimed:

1. A method of administering pirfenidone to a human patient in need thereof comprising administering to the patient (1) food and (2) a pharmaceutical composition comprising a therapeutically effective dose of pirfenidone, wherein the ratio of the mean maximum plasma concentration of pirfenidone when said pharmaceutical composition is administered to the patient under fed conditions (C_{max(fed)}) to the mean maximum plasma concentration of pirfenidone when said pharmaceutical composition is administered to said patient under fasted conditions (C_{max(fasted)}) is about 0.35 to about 0.7.

2. The method of claim 1, wherein the total daily amount of pirfenidone administered to the patient is about 2403 mg.

3. The method of claim 1, wherein the area under the curve of the absorption curve of pirfenidone when administered under fed conditions (AUC_{fed}) is at least about 80% of the area under the curve of the absorption curve of pirfenidone when administered under fasted conditions (AUC_{fasted}).

4. The method of claim 1, wherein the ratio of the mean absorption half life of pirfenidone when said pharmaceutical composition is administered to the patient under fed conditions (t_{1/2, abs(fed)}) to the mean absorption half life of pirfenidone when said pharmaceutical composition is administered to the patient under fasted conditions t_{1/2, abs(fasted)}) is about 2.5 to about 3.5.

5. The method of claim 4, wherein the area under the curve of the absorption curve of pirfenidone when administered under fed conditions (AUC_{fed}) is at least about 80% of the area under the curve of the absorption curve of pirfenidone when administered under fasted conditions (AUC_{fasted}).

6. The method of claim 3, wherein the total daily amount of pirfenidone administered to the patient is about 2403 mg.

7. The method of claim 1, wherein the administration of the food is within one hour of the administration of the pirfenidone.

8. The method of claim 1, further comprising advising the patient that taking pirfenidone under fed conditions may reduce the incidence of adverse events resulting from pirfenidone therapy in comparison to taking pirfenidone under fasted conditions.

9. The method of claim 8, wherein the adverse event comprises one of more of dizziness, somnolence, or headaches.

10. A method of providing pirfenidone therapy to a human patient in need thereof comprising advising the patient that the mean maximum plasma concentration of pirfenidone when administered under fed conditions (C_{max(fed)}) is lower than the mean maximum plasma concentration of pirfenidone when administered under fasted conditions (C_{max(fasted)}), wherein the ratio of C_{max(fed)}:C_{max(fasted)} is about 0.35 to about 0.7.

11. The method of claim 10, wherein the area under the curve of the absorption curve of pirfenidone when administered under fed conditions (AUC_{fed}) is at least about 80% of the area under the curve of the absorption curve of pirfenidone when administered under fasted conditions (AUC_{fasted}).

12. The method of claim 10 further comprising advising the patient that the mean absorption half-life of pirfenidone under fed conditions (t_{1/2, abs(fed)}) is longer than the mean absorption half-life of pirfenidone under fasted conditions (t_{1/2, abs(fasted)}), wherein the ratio t_{1/2, abs(fed)}:t_{1/2, abs(fasted)} is about 2.5 to about 3.5.

13. The method of claim 12, wherein the area under the curve of the absorption curve of pirfenidone when administered under fed conditions (AUC_{fed}) is at least about 80% of the area under the curve of the absorption curve of pirfenidone when administered under fasted conditions (AUC_{fasted}).

14. The method of claim 11 further comprising advising the patient that taking pirfenidone under fed conditions may reduce the incidence of adverse events resulting from pirfenidone therapy in comparison to taking pirfenidone under fasted conditions.

15. The method of claim 14, wherein the adverse event comprises one or more of dizziness, somnolence, or headaches.

16. The method of claim 10, further advising the patient to take a total daily amount of pirfenidone of about 2403 mg.
